(19) United States
(12) Patent Application Publication
(10) Pub. No.: US 2022/0301660 A1
(43) Pub. Date: Sep. 22, 2022

(54) METHOD AND DEVICE FOR MULTIPLE TRANSITION MONITORING

(71) Applicant: Roche Diagnostics Operations, lnc., Indianapolis, IN (US)

(72) Inventors: Florian Schweinberger, Muenchen (DE); Aart Pieter Van Doorn, Muenchen (DE)

(73) Assignee: Roche Diagnostics Operations, lnc., Indianapolis, IN (US)

(21) Appl. No.: 17/805,899

(22) Filed: Jun. 8, 2022

Related U.S. Application Data
(63) Continuation of application No. PCT/EP2020/086476, filed on Dec. 16, 2020.

(30) Foreign Application Priority Data
Dec. 17, 2019 (EP) 19216968.8

(51) Int. Cl. G16B 40/10; G06K 9/00

(52) U.S. Cl. CPC G16B 40/10; G06K 9/00543; G06K 9/0053

(57) ABSTRACT

A method for multiple transition monitoring using a liquid chromatography mass spectrometry device is disclosed and comprises determining at least one data set from at least one data base, the data set comprising at least one reference measurement of at least one transition of at least one analyte with the liquid chromatography mass spectrometry device; determining at least one reference peak information of the transition of the analyte using an initial setting of a measurement window, wherein the measurement window is defined by a time frame of retention times; determining an actual setting of the measurement window considering the reference peak information, wherein the determining comprises adjusting the time frame; measuring the transition of the analyte with the liquid chromatography mass spectrometry device and determining a measured peak information of the transition of the analyte using the actual setting of the measurement window.

### CROSS-REFERENCE TO RELATED APPLICATIONS

This application is a continuation of International Patent Application No. PCT/EP2020/086476, filed 16 Dec. 2020, which claims priority to European Patent Application No. 19216968.8, filed 17 Dec. 2019, the disclosures of which are hereby incorporated by reference in their entirety.

### TECHNICAL FIELD

The present disclosure relates to a method and a device for multiple transition monitoring using mass spectrometry techniques, specifically liquid chromatography and mass spectrometry.

### BACKGROUND

Multiple transition monitoring (MRM) during one liquid chromatography run reduces dwell times for each transition with increasing number of observer MRM. Known methods such as scheduled MRMs use only the times frames in a liquid chromatography run that is relevant to a certain transition. For example, analyte X with MRM Y is resulting in peak Z at time x and, thus, only a certain time of relevance such as x−15 s to x+15 s may be recorded. Usually a certain time frame is defined with respect to a certain time before and after the peak maximum of the peak, usually referred to as the retention time.

Scheduled MRM in particular works well for very defined measurements and/or analyte mixtures with high concentrations. Despite the advantages of scheduled MRM, however, there is growing interest for maximizing column lifetimes, which may go hand in hand with significant peak shifts such that scheduled MRM with a fixed time frame for peak recording may lead to non reliable and incorrect results.

### SUMMARY

Although the embodiments of the present disclosure are not limited to specific advantages or functionality, it is noted that the present disclosure provides a method and a device for multiple transition monitoring, which avoid the above-described disadvantages of known methods and devices. In particular, the method and the device allow reliable and correct multiple transition monitoring even in case of retention time shifts due to column aging, capillary exchanges, solvent composition inaccuracies, and other factors. Moreover, the method and the device allow lowering limit of quantification, specifically for critical analytes.

In accordance with one embodiment, a method for multiple transition monitoring using a liquid chromatography mass spectrometry device is disclosed, the method comprising the following steps: a) determining at least one data set from at least one data base, the data set comprising at least one reference measurement of at least one transition of at least one analyte with the liquid chromatography mass spectrometry device; b) determining at least one reference peak information of the transition of the analyte using an initial setting of a measurement window, wherein the initial setting is a setting of the measurement window which is used for determining the reference peak information, wherein the measurement window is defined by a time frame of retention times; c) determining an actual setting of the measurement window considering the reference peak information, wherein the determining comprises adjusting the time frame, wherein the actual setting is a setting of the measurement window determined by considering the reference peak information determined in step b); d) measuring the transition of the analyte with the liquid chromatography mass spectrometry device and determining a measured peak information of the transition of the analyte using the actual setting of the measurement window.

In accordance with another embodiment, a device for multiple transition monitoring of at least one analyte in a sample is disclosed comprising: at least one liquid chromatography mass spectrometer device configured for multiple transition monitoring; at least one data base configured for storing at least one data set comprising at least one reference measurement of at least one transition of at least one analyte; at least one evaluation device, wherein the evaluation device is configured for determining at least one reference peak information of the transition of the analyte using an initial setting of a measurement window, wherein the initial setting is a setting of the measurement window which is used for determining the reference peak information, wherein the measurement window is defined by a time frame of retention times, wherein the evaluation device is configured for determining an actual setting of the measurement window considering the reference peak information, wherein the determining comprises adjusting the time frame, wherein the actual setting is a setting of the measurement window determined by considering the determined reference peak information, wherein the evaluation device is configured for determining a measured peak information of the transition of the analyte of a subsequent measurement of the transition of the analyte with the liquid chromatography mass spectrometry device using the actual setting of the measurement window.

These and other features and advantages of the embodiments of the present disclosure will be more fully understood from the following detailed description taken together with the accompanying claims. It is noted that the scope of the claims is defined by the recitations therein and not by the specific discussions of features and advantages set forth in the present description.

### BRIEF DESCRIPTION OF THE DRAWINGS

The following detailed description of the embodiments of the present disclosure can be best understood when read in conjunction with the following drawings, where like structure is indicated with like reference numbers and in which:

FIGS. 1 A and 1 B show a flow chart of a method in accordance with an embodiment of the present disclosure;

FIGS. 2 A and 2 B show a comparison of a usual approach (FIG. 2 A) and the method in accordance with an embodiment of the present disclosure (FIG. 2 B);

FIGS. 3 A and 3 B show a further comparison of measurement windows of a usual approach (FIG. 3 A) and of the method in accordance with an embodiment of the present disclosure (FIG. 3 B); and

FIG. 4 shows a device for multiple transition monitoring in accordance with an embodiment of the present disclosure.

Skilled artisans appreciate that elements in the figures are illustrated for simplicity and clarity and have not been drawn to scale. For example, dimensions of some of the elements in the figures may be exaggerated relative to other elements to help improve understanding of the embodiment(s) of the present disclosure.

### DETAILED DESCRIPTION OF THE DISCLOSURE

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The present disclosure may, as the skilled person will recognize, be performed by using alternative features. Similarly, features introduced by “in an embodiment of the present disclosure” or similar expressions are intended to be optional features, without any restriction regarding alternative embodiments of the present disclosure, without any restrictions regarding the scope of the present disclosure and without any restriction regarding the possibility of combining the features introduced in such way with other optional or non-optional features of the present disclosure.

The term “multiple transition monitoring”, also denoted multiple reaction monitoring (MRM), as used herein is a broad term and is to be given its ordinary and customary meaning to a person of ordinary skill in the art and is not to be limited to a special or customized meaning. The term specifically may refer, without limitation, to a method used in mass spectrometry, specifically in tandem mass spectrometry, in which multiple product ions from one or more precursor ions are monitored. As used herein, the term “monitored” is a broad term and is to be given its ordinary and customary meaning to a person of ordinary skill in the art and is not to be limited to a special or customized meaning. The term specifically may refer, without limitation, to determining and/or detecting of multiple product ions.

As used herein, the term “liquid chromatography mass spectrometry device” is a broad term and is to be given its ordinary and customary meaning to a person of ordinary skill in the art and is not to be limited to a special or customized meaning. The term specifically may refer, without limitation, to a combination of liquid chromatography with mass spectrometry. The liquid chromatography mass spectrometry device may be or may comprise at least one high-performance liquid chromatography (HPLC) device or at least one micro liquid chromatography (AC) device. The liquid chromatography mass spectrometry device may comprise a liquid chromatography (LC) device and a mass spectrometry (MS) device, wherein the LC device and the MS are coupled via at least one interface.

As used herein, the term “liquid chromatography (LC) device” is a broad term and is to be given its ordinary and customary meaning to a person of ordinary skill in the art and is not to be limited to a special or customized meaning. The term specifically may refer, without limitation, to an analytical module configured to separate one or more analytes of interest of a sample from other components of the sample for detection of the one or more analytes with the mass spectrometry device. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analytes of interest. The LC column may be exchangeable, for example after a predefined or pre-determined time and/or number of runs, and/or other suitable counters. For example, the LC column may be exchanged if one or more thresholds of one or more of a volume of solvent, a number of switching event of valves, a number of runs, a number of injections, a number of samples, a number of samples of a certain type, an LC pressure/curves are reached. As used herein, the term “mass spectrometry device” is a broad term and is to be given its ordinary and customary meaning to a person of ordinary skill in the art and is not to be limited to a special or customized meaning. The term specifically may refer, without limitation, to a mass analyzer configured for detecting at least one analyte based on mass to charge ratio. The mass spectrometry device may be or may comprise at least one quadrupole mass spectrometry device. The interface coupling the LC device and the MS may comprise at least one ionization source configured for generating of molecular ions and for transferring of the molecular ions into the gas phase.

As used herein, the term “sample” is a broad term and is to be given its ordinary and customary meaning to a person of ordinary skill in the art and is not to be limited to a special or customized meaning. The term specifically may refer, without limitation, to an arbitrary sample such as a biological sample, also called test sample, a quality control sample, an internal standard sample. The sample may comprise one or more analytes of interest. For example, the test sample may be selected from the group consisting of: a physiological fluid, including blood, serum, plasma, saliva, ocular lens fluid, cerebral spinal fluid, sweat, urine, milk, ascites fluid, mucous, synovial fluid, peritoneal fluid, amniotic fluid, tissue, cells or the like. The sample may be used directly as obtained from the respective source or may be subject of a pretreatment and/or sample preparation workflow. For example, the sample may be pretreated by adding an internal standard and/or by being diluted with another solution and/or by having being mixed with reagents or the like. For example, analytes of interest may be vitamin D, drugs of abuse, therapeutic drugs, hormones, and metabolites in general. The quality control sample may be a sample that mimics the test sample, and that contains known values of one or more quality control substances. The quality control substance may be identical to the analyte of interest or may be an analyte which generates by reaction or derivatization an analyte identical to the analyte of interest and/or may be an analyte of which the concentration is known and/or may be a substance which mimics the analyte of interest or that can be otherwise correlated to a certain analyte of interest. The internal standard sample may be a sample comprising at least one internal standard substance with a known concentration. For further details with respect to the sample, reference is made e.g., to EP 3 425 369 A1, the full disclosure is included herewith by reference. Other analytes of interest are possible.

The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlapping fashion. The method may comprise further method steps which are not listed.

The method comprises the following steps:
a) determining at least one data set from at least one data base, the data set comprising at least one reference measurement of at least one transition of at least one analyte with the liquid chromatography mass spectrometry device;
b) determining at least one reference peak information of the transition of the analyte using an initial setting of a measurement window, wherein the measurement window is defined by a time frame of retention times;
c) determining an actual setting of the measurement window considering the reference peak information, wherein the determining comprises adjusting the time frame;
d) measuring the transition of the analyte with the liquid chromatography mass spectrometry device and determining a measured peak information of the transition of the analyte using the actual setting of the measurement window.

As used herein, the term “data set” is a broad term and is to be given its ordinary and customary meaning to a person of ordinary skill in the art and is not to be limited to a special or customized meaning. The term specifically may refer, without limitation, to stored and/or deposited information about at least one previous MRM measurement such as from previous runs. The information about the previous MRM measurement may comprise at least one chromatogram and/or at least one information evaluated from the chromatogram such as peak maximum, retention time, peak start time, peak end time, peak width, in particular the full width half maximum, peak shape, tailing factor, and/or any type of peak fitting and filtering. The tailing factor T may be determined by T=W_{0.05}/(2d), wherein W_{0.05} is the peak width at 0.05 of the peak height and d is a distance between a perpendicular line through the peak maximum and a leading edge of the peak at 0.05 of the peak height. As used herein, the term “data base” is a broad term and is to be given its ordinary and customary meaning to a person of ordinary skill in the art and is not to be limited to a special or customized meaning. The term specifically may refer, without limitation, to a collection of data comprising the at least one data set. The data base may comprise at least one table and/or at least one look-up table in which the at least one data set is stored. The data base may comprise at least one storage unit configured to store the data set. As used herein, the term “determining at least one data set from at least one data base” is a broad term and is to be given its ordinary and customary meaning to a person of ordinary skill in the art and is not to be limited to a special or customized meaning. The term specifically may refer, without limitation, to access the data base and to retrieve the data set from the data base.

As used herein, the term “reference measurement” is a broad term and is to be given its ordinary and customary meaning to a person of ordinary skill in the art and is not to be limited to a special or customized meaning. The term specifically may refer, without limitation, to at least one previous MRM measurement such as a MRM measurement of a previous run and/or at least one prediction about a MRM measurement. The reference measurement may comprise or may be at least one known MRM transition. The reference measurement may be at least one measurement of at least one quality control sample acquired during a previous quality control run and/or at least one measurement of at least one internal standard sample acquired during a previous internal standard sample run and/or at least one measurement of the test sample acquired during a previous run. The reference measurement may be at least one measurement acquired and/or determined and/or measured in the same way and under the same or at least similar and/or comparable conditions as the measurement of the actual test sample. The reference measurements and the measuring of the transition of the analyte may be performed under substantially the same conditions. For example, the reference measurement and the measuring of the transition of the analyte may be performed under constant chromatographic conditions, specifically with the same LC column and eluents. The method may apply to known compounds and well-known conditions. The method may however also comprise predicting at least one reference measurement, specifically in case of changes of gradients. The predicting may comprise considering aging of LC column, capillary exchanges, solvent composition inaccuracies, and other factors.

As used herein, the term “reference peak information” is a broad term and is to be given its ordinary and customary meaning to a person of ordinary skill in the art and is not to be limited to a special or customized meaning. The term specifically may refer, without limitation, to at least one information of a peak, i.e., a local maximum, of the chromatogram corresponding and/or relating to the analyte of interest of the reference measurement which is suitable to limit the relevant time frame for measurement of the analyte of interest. The reference peak information may comprise one or more of: peak maximum, retention time, peak start time, peak end time, peak width, in particular the full width half maximum, peak shape, tailing factor and/or any type of peak fitting and filtering. The determining of the reference peak information may comprise evaluating the reference measurement. The evaluating may comprise performing at least one data analysis comprising performing at least one peak finding algorithm and/or performing at least one peak fitting algorithm. The evaluating may comprise one or more of checking of raw data, preprocessing, smoothening, background reduction or removal, peak detection, peak integration.

As used herein, the term “measurement window” is a broad term and is to be given its ordinary and customary meaning to a person of ordinary skill in the art and is not to be limited to a special or customized meaning. The term specifically may refer, without limitation, to a time frame in which the measurement of the analyte of interest is performed. The measurement window is defined by a time frame of retention times. Limiting the measurement of the analyte of interest to a certain pre-defined time frame is generally known. As used herein, the term “setting” of the measurement window is a broad term and is to be given its ordinary and customary meaning to a person of ordinary skill in the art and is not to be limited to a special or customized meaning. The term specifically may refer, without limitation, to values for one or both limits of the measurement window. Specifically, the setting of the measurement window may comprise one or both of a value for a lower limit of the measurement window, i.e., a retention time at which the measurement starts, and a value for an upper limit of the measurement window, i.e., a retention time at which the measurement stops.

The term “initial setting” is a broad term and is to be given its ordinary and customary meaning to a person of ordinary skill in the art and is not to be limited to a special or customized meaning. The term specifically may refer, without limitation, to a setting of the measurement window which is used for determining the reference peak information. The initial setting may be pre-determined and/or pre-defined. For example, in case of a first measurement after change of a column of the LC device, the initial setting may be a default setting which may be deposited in the data base. The method may comprise at least one initial calibration step, wherein in the initial calibration step the reference measurement and/or the initial setting may be determined. The initial calibration step may be performed during and/or subsequent to at least one quality control run of the liquid chromatography mass spectrometry device and/or during and/or subsequent to at least one internal standard samples run. A start of the initial calibration step may be triggered by changing of a column of the liquid chromatography mass spectrometry device and/or after a predefined or pre-determined time and/or after a predefined or predetermined number of runs, or other suitable counters. The term “trigger” as used herein, may refer to either an automatic procedure that is initiated and executed automatically or a warning generated for and prompting a user to manually set the setting to the initial setting. In case of performing the method repeatedly, the initial setting may be a setting of the measurement window determined from the measurement of at least one prior run or a plurality of prior runs, such as a mean value for the limits of the time frame.

The term “actual setting” is a broad term and is to be given its ordinary and customary meaning to a person of ordinary skill in the art and is not to be limited to a special or customized meaning. The term specifically may refer, without limitation, to a setting of the measurement window determined by considering the reference peak information determined in step b). In addition, to considering the reference peak information of only the preceding measurement, the actual setting may be determined considering the preceding measurement or a plurality of preceding measurements. The determining of the actual setting may comprise evaluating the reference peak information and thereby determining a lower and/or an upper limit of the measurement window. Specifically, at least one automated analysis of retention times may be performed. Moreover, the measurement window may be automatically reassigned. The actual setting may be determined and/or calculated based on expected retention time and tailing factor. The actual setting may be determined by comparing peak, in particular signal intensity, and background. For example, signal intensity and background may be compared by defining and/or using at least one threshold value at which the peak starts and/or at least one threshold value at which the peak ends. The actual setting may be determined by making a prediction based on a plurality of datasets.

The initial setting may comprise a broader time frame of retention times compared to the actual setting and/or the actual setting may be shifted in retention time compared to the initial setting. In the latter case the width of the measurement window may be maintained. Specifically, the initial setting of the measurement window may be selected so broad such that it is ensured that the peak corresponding to the analyte of interest lies within the time frame. Subsequently the initial setting of the measurement window may be optimized in view of measurement results which allows for reducing width of the measurement window and/or for positioning the measurement window, specifically to take into account changes of the LC column such as due to aging or other changes. The reference measurement may comprise or may be a known MRM transition, wherein the method comprises optimizing their measurement. The term “determining the actual setting” is a broad term and is to be given its ordinary and customary meaning to a person of ordinary skill in the art and is not to be limited to a special or customized meaning. The term specifically may refer, without limitation, to adapting and/or changing the initial setting of the measurement window depending on at least one subsequent measurement. The timing of the measurement may be fixed. However, due to variation of the peak position as a result of ongoing analysis, specifically in case of repeatedly performing method steps a) to d), the retention time may shift and may be adapted based on prior measurements. The method may comprise adjustment of the MRM scheduled timing as a function of changing parameters over time. Thus, the optimized retention time may be used to optimize scheduled MRM measurements and thus freeing up time for more MRM transitions.

The method comprises in step d) measuring the transition of the analyte within a sample with the liquid chromatography mass spectrometry device. The measurement may be triggered by a user, e.g., by entering at least one input to at least one human-machine-interface of the liquid chromatography mass spectrometry device.

The actual setting of the measurement window is used for determining the measured peak information of the transition of the analyte. As used herein, the term “measured peak information” is a broad term and is to be given its ordinary and customary meaning to a person of ordinary skill in the art and is not to be limited to a special or customized meaning. The term specifically may refer, without limitation, to at least one information of a peak of the chromatogram measured in step d) corresponding and/or relating to the analyte of interest using the actual setting of the measurement window. The measured peak information comprises one or more of: peak maximum, retention time, peak start time, peak end time, peak width, in particular the full width half maximum, peak shape, tailing factor. The determining of the measured peak information may comprise evaluating the actual measurement. The evaluating may comprise performing at least one data analysis comprising performing at least one peak finding algorithm and/or performing at least one peak fitting algorithm.

The method further may comprise updating the data set by adding the measurement of the transition of the analyte to the data set. Specifically, in case the method steps a) to d) are performed repeatedly, the data set may be updated after performing step d) such that the subsequent step a) is performed using an updated initial setting. Thus, the updating may be performed permanently.

Method steps a) to d) may be performed repeatedly. In step a) the most recent measurement may be used as reference measurement. In step b) the reference peak information of the transition of the analyte may be determined using the most recently determined actual setting as initial setting. After a plurality of repetitions of methods steps a) to d), in step a) a mean value of a plurality of preceding measurements may be used as reference measurement. The mean value may be determined by using at least five preceding measurements. Additionally or alternatively, in particular in case of larger changes between runs, a moving average of a plurality of preceding measurements may be used as reference measurement. Additionally or alternatively, a maximum amount of preceding measurements for calculating the mean value may be limited. Using more than one measurement may ensure that the reference data is corrected with respect to outliers or sample influence.

A width of the measurement window may narrow with number of repetitions. Thus, the measurement window becomes even better and/or more beneficial for clearing up more MS detection window. The detection window may be a time frame in which the mass spectrometry device has to perform a measurement of a sample. Specifically, the detection window may be the maximum time possible between two consecutive sample inputs in the LC column.

The method may further comprise at least one in-situ adjustment step. The in-situ adjustment step may be performed during step d). In the in-situ adjustment step intensity of the transition of the analyte may be monitored during the measurement and compared to at least one predetermined or predefined threshold level. The liquid chromatography mass spectrometry device may comprise at least one further data base configured for storing at least one definition of threshold levels and/or threshold values. For example, the at least one threshold level and/or the at least one threshold value may be defined by percentual change of signal intensity to background. Additionally or alternatively, at least one absolute value may be used as threshold for determining exceedance or undershoot. The further data base may be configured to receive input information from the data base such as values for the threshold levels. Thus, the threshold levels may be data driven. If the intensity falls below the predefined threshold level acquisition of the transition may be stopped. The predetermined or predefined threshold level may be defined by a factor X times the signal-to-noise ratio which is also known from the start. The in-situ adjustment step may be implemented as a feedback loop with automated live adjustment of measurement parameter. For example, a measurement, i.e., a specific MRM, may start at a time known on the basis of previous measurements. A run time of the measurement may be determined on the basis of an actual measured intensity of this MRM. In case the intensity falls below the predetermined or predefined threshold level the acquisition of that MRM may be stopped and may allow to free dwell time for other MRMs running at the same time. This approach may be reliable for well articulated peaks. However, problems may arise when signal to noise is low, e.g., a small peak with high variation in individual signal. For these cases it may be advantageous that the predetermined or predefined threshold level may be given by an end of a fit curve such as a Gaussian curve. The fit curve may allow defining the peak region such as start of the peak and end of the peak. In particular, a certain peak height at the end of the peak, denoted as end of the fit curve, may be used as threshold level. In principle, use of a fit curve during measurement is problematic since the full signal is not present at this stage. However, a fit curve such as a Gaussian curve can be used since the fit parameters of the Gaussian or other fit curve may be determined from the one or more preceding measurements. This may allow determining all parameters of the fit curve with only a single iteration. Additionally or alternatively, the fit curve may be determined from a measurement of an internal standard. This may allow accelerating the fitting procedure. Additionally or alternatively, a combination of fit results of different peaks of the same analyte may be used to enhance robustness of the fit result. The fit curve, in particular of a Gaussian, may be independent or less dependent from background and, thus, advantageous even at high noise.

The method steps b) to c) and in step d) the determining of the measured peak information of the transition of the analyte may be performed by at least one computer. Specifically, the method steps b) to c) and in step d) the determining of the measured peak information of the transition of the analyte may be performed fully automatically. The method specifically may fully or partially be computer-implemented, specifically on a computer of a device for multiple transition monitoring, such as a processor.

The method may comprise compensating for column aging and/or for further impacts such as capillary exchanges, solvent composition inaccuracies, and the like.

In a further aspect, a computer program including computer-executable instructions for performing the method according to any one of the embodiments as described herein is disclosed, specifically method steps a) to c) and determining of the measured peak information in step d), when the program is executed on a computer or computer network, specifically a processor of the device for multiple transition monitoring.

Thus, generally speaking, disclosed and proposed herein is a computer program including computer-executable instructions for performing the method according to the present disclosure in one or more of the embodiments enclosed herein when the program is executed on a computer or computer network. Specifically, the computer program may be stored on a computer-readable data carrier. Thus, specifically, one, more than one or even all of the method steps as indicated above may be performed by using a computer or a computer network, preferably by using a computer program. The computer specifically may be fully or partially integrated into the device for multiple transition monitoring, and the computer programs specifically may be embodied as a software. Alternatively, however, at least part of the computer may also be located outside the device for multiple transition monitoring.

Further disclosed and proposed herein is a computer program product having program code means, in order to perform the method according to the present disclosure in one or more of the embodiments enclosed herein when the program is executed on a computer or computer network, e.g., one or more of the method steps mentioned above. Specifically, the program code means may be stored on a computer-readable data carrier.

Further disclosed and proposed herein is a data carrier having a data structure stored thereon, which, after loading into a computer or computer network, such as into a working memory or main memory of the computer or computer network, may execute the method according to one or more of the embodiments disclosed herein, specifically one or more of the method steps mentioned above.

Further disclosed and proposed herein is a computer program product with program code means stored on a machine-readable carrier, in order to perform the method according to one or more of the embodiments disclosed herein, when the program is executed on a computer or computer network, specifically one or more of the method steps mentioned above. As used herein, a computer program product refers to the program as a tradable product. The product may generally exist in an arbitrary format, such as in a paper format, or on a computer-readable data carrier. Specifically, the computer program product may be distributed over a data network.

Finally, disclosed and proposed herein is a modulated data signal which contains instructions readable by a computer system or computer network, for performing the method according to one or more of the embodiments disclosed herein, specifically one or more of the method steps mentioned above.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In a further aspect of the present disclosure, a device for multiple transition monitoring of at least one analyte in a sample is disclosed. The device comprises
at least one liquid chromatography mass spectrometer device configured for multiple transition monitoring;
at least one data base configured for storing at least one data set comprising at least one reference measurement of at least one transition of at least one analyte;
at least one evaluation device, wherein the evaluation device is configured for determining at least one reference peak information of the transition of the analyte using an initial setting of a measurement window, wherein the measurement window is defined by a time frame of retention times, wherein the evaluation device is configured for determining an actual setting of the measurement window considering the reference peak information, wherein the determining comprises adjusting the time frame, wherein the evaluation device is configured for determining a measured peak information of the transition of the analyte of a subsequent measurement of the transition of the analyte with the liquid chromatography mass spectrometry device using the actual setting of the measurement window.

The device may be configured to perform the method according to any one of the preceding embodiments. For most of the terms used herein and possible definitions, reference may be made to the description of the methods above.

As further used herein, the term “evaluation device” generally refers to an arbitrary device adapted to perform the method steps as described above, preferably by using at least one data processing device and, more preferably, by using at least one processor and/or at least one application-specific integrated circuit. Thus, as an example, the at least one evaluation device may comprise at least one data processing device having a software code stored thereon comprising a number of computer commands. The evaluation device may provide one or more hardware elements for performing one or more of the named operations and/or may provide one or more processors with software running thereon for performing one or more of the method steps.

The methods and devices according to the present disclosure may provide a large number of advantages over known methods and devices for multiple transition monitoring. Thus, specifically, dynamic MRMs on the basis of previous runs may free up valuable channel time and consequently increase sampling rate or time for analytes. This is particularly beneficial for analytes that should be measured with high sensitivity. Influences that may be caused by changes of components over their lifetime, e.g., column aging, and/or further impacts such as capillary exchanges, solvent composition inaccuracies, and other factors, and resulting retention time shifts or changes and impact on spray stabilization, can be compensated within the boundary of the available time window. Deviations arising from column batch to batch variations can be compensated, too. The method does not require hardware changes but can be implemented as fast and powerful in-situ algorithms.

Summarizing and without excluding further possible embodiments, the following embodiments may be envisaged:

Embodiment 1: Method for multiple transition monitoring using a liquid chromatography mass spectrometry device, the method comprises the following steps:
a) determining at least one data set from at least one data base, the data set comprising at least one reference measurement of at least one transition of at least one analyte with the liquid chromatography mass spectrometry device;
b) determining at least one reference peak information of the transition of the analyte using an initial setting of a measurement window, wherein the measurement window is defined by a time frame of retention times;
c) determining an actual setting of the measurement window considering the reference peak information, wherein the determining comprises adjusting the time frame;
d) measuring the transition of the analyte with the liquid chromatography mass spectrometry device and determining a measured peak information of the transition of the analyte using the actual setting of the measurement window.

Embodiment 2: The method according to the preceding embodiment, wherein the method further comprises updating the data set by adding the measurement of the transition of the analyte to the data set.

Embodiment 3: The method according to the preceding embodiment, wherein method steps a) to d) are performed repeatedly, wherein in step a) the most recent measurement is used as reference measurement, wherein in step b) the reference peak information of the transition of the analyte is determined using the most recently determined actual setting as initial setting.

Embodiment 4: The method according to the pre-preceding embodiment, wherein in step a) a mean value of a plurality of preceding measurements is used as reference measurement.

Embodiment 5: The method according to the preceding embodiment, wherein the mean value is determined by using at least five preceding measurements.

Embodiment 6: The method according to any one of the two preceding embodiments, wherein a maximum amount of preceding measurements for calculating the mean value is limited.

Embodiment 7: The method according to embodiment 2, wherein in step a) a moving average of a plurality of preceding measurements is used as reference measurement.

Embodiment 8: The method according to any one of the preceding embodiments, wherein the method further comprises at least one in-situ adjustment step, wherein the in-situ adjustment step is performed during step d), wherein in the in-situ adjustment step intensity of the transition of the analyte is monitored during the measurement and compared to at least one predetermined or predefined threshold level, wherein if the intensity falls below the predefined threshold level acquisition of the transition is stopped.

Embodiment 9: The method according to the preceding embodiment, wherein the at least one threshold level and/or the at least one threshold value is defined by percentual change of signal intensity to background and/or at least one absolute value is used as threshold for determining exceedance or undershoot.

Embodiment 10: The method according to the pre-preceding embodiment, wherein the predetermined or predefined threshold level is given by an end of a fit curve such as a Gaussian curve, wherein the parameters of the fit curve are determined from one or more preceding measurements, and/or wherein the fit curve is determined from a measurement of an internal standard, and/or wherein a combination of fit results of different peaks of the same analyte are used to enhance robustness of the fit result.

Embodiment 11: The method according to any one of the preceding embodiments, wherein the reference peak information and/or the measured peak information comprise one or more of: peak maximum, retention time, peak start time, peak end time, peak width, in particular the full width half maximum, peak shape, tailing factor, and/or any type of peak fitting and filtering.

Embodiment 12: The method according to any one of the preceding embodiments, wherein the method comprises at least one initial calibration step, wherein in the initial calibration step the reference measurement and/or the initial setting are determined.

Embodiment 13: The method according to the preceding embodiment, wherein the initial calibration step is performed during and/or subsequent to at least one quality control run of the liquid chromatography mass spectrometry device and/or during and/or subsequent to at least one internal standard samples run.

Embodiment 14: The method according to any one of the two preceding embodiments, wherein a start of the initial calibration step is triggered by changing of a column of the liquid chromatography mass spectrometry device and/or after a predefined or pre-determined time and/or after a predefined or predetermined number of runs, or other suitable counters.

Embodiment 15: The method according to any one of the preceding embodiments, wherein the initial setting comprises a broad time frame of retention times compared to the actual setting.

Embodiment 16: The method according to any one of the preceding embodiments, wherein the method comprises repeating method steps a) to d), wherein a width of the measurement window narrows with number of repetitions.

Embodiment 17: The method according to any one of the preceding embodiments, wherein the reference measurements and the measuring of the transition of the analyte are performed under substantially the same conditions.

Embodiment 18: The method according to any one of the preceding embodiments, wherein the method steps b) to c) and in step d) the determining of the measured peak information of the transition of the analyte are performed by at least one computer.

Embodiment 19: The method according to any one of the preceding embodiments, wherein the method comprises compensating for column aging and/or for further impacts such as capillary exchanges, solvent composition inaccuracies, and the like.

Embodiment 20: The method according to any one of the preceding embodiments, wherein the actual setting is determined and/or calculated based on expected retention time and tailing factor, and/or wherein the actual setting is determined by comparing peak and background, and/or wherein the actual setting is determined by making a prediction based on a plurality of datasets.

Embodiment 21: A device for multiple transition monitoring of at least one analyte in a sample comprising:
at least one liquid chromatography mass spectrometer device configured for multiple transition monitoring;
at least one data base configured for storing at least one data set comprising at least one reference measurement of at least one transition of at least one analyte;
at least one evaluation device, wherein the evaluation device is configured for determining at least one reference peak information of the transition of the analyte using an initial setting of a measurement window, wherein the measurement window is defined by a time frame of retention times, wherein the evaluation device is configured for determining an actual setting of the measurement window considering the reference peak information, wherein the determining comprises adjusting the time frame, wherein the evaluation device is configured for determining a measured peak information of the transition of the analyte of a subsequent measurement of the transition of the analyte with the liquid chromatography mass spectrometry device using the actual setting of the measurement window.

Embodiment 22: The device according to the preceding embodiment, wherein the device is configured to perform the method according to any one of the preceding embodiments referring to a method.

In order that the embodiments of the present disclosure may be more readily understood, reference is made to the following examples, which are intended to illustrate the disclosure, but not limit the scope thereof.

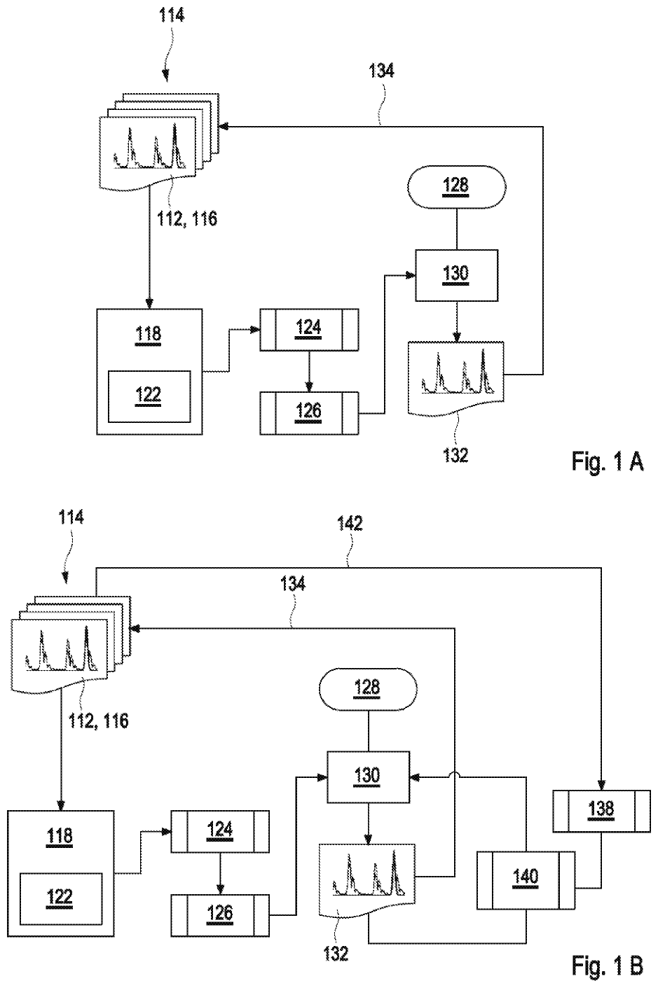

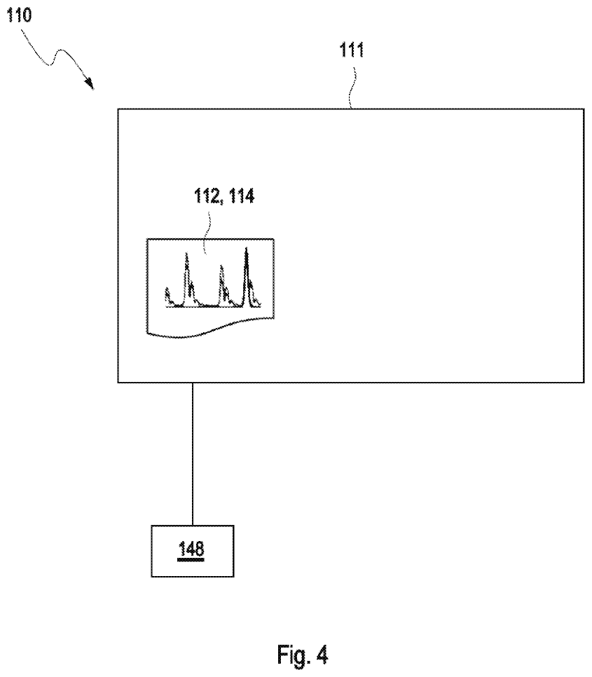

FIG. 1 A shows a flow chart of a method for multiple transition monitoring using a liquid chromatography mass spectrometry device 111, an embodiment of which is shown in FIG. 4, according to the present disclosure.

The method comprises in step a) determining at least one data set 112 from at least one data base 114. The data set 112 comprises at least one reference measurement 116 of at least one transition of at least one analyte with the liquid chromatography mass spectrometry device 111. The data set 112 may be and/or may comprise stored and/or deposited information about at least one previous MRM measurement such as from previous runs. The information about the previous MRM measurement may comprise at least one chromatogram and/or at least one information evaluated from the chromatogram such as peak maximum, retention time, peak start time, peak end time, peak width, in particular the full width half maximum, peak shape, tailing factor, and/or any type of peak fitting and filtering. The data base 114 may comprise at least one table and/or at least one look-up table in which the at least one data set 112 is stored. The data base may comprise at least one storage unit configured to store the data set.

The reference measurement may comprise or may be at least one known MRM transition. The reference measurement may be at least one measurement of at least one quality control sample acquired during a previous quality control run and/or at least one measurement of at least one internal standard sample acquired during a previous internal standard sample run and/or at least one measurement of the test sample acquired during a previous run. The reference measurement may be at least one measurement acquired and/or determined and/or measured in the same way and under the same or at least similar and/or comparable conditions as the measurement of the actual test sample. The reference measurements and the measuring of the transition of the analyte may be performed under substantially the same conditions. For example, the reference measurement and the measuring of the transition of the analyte may be performed under constant chromatographic conditions, specifically with the same LC column and eluent. The method may apply to known compounds and well-known conditions. The method may comprise predicting at least one reference measurement, specifically in case of changes of gradients. The predicting may comprise considering aging of LC column, capillary exchanges, solvent composition inaccuracies, and other factors.

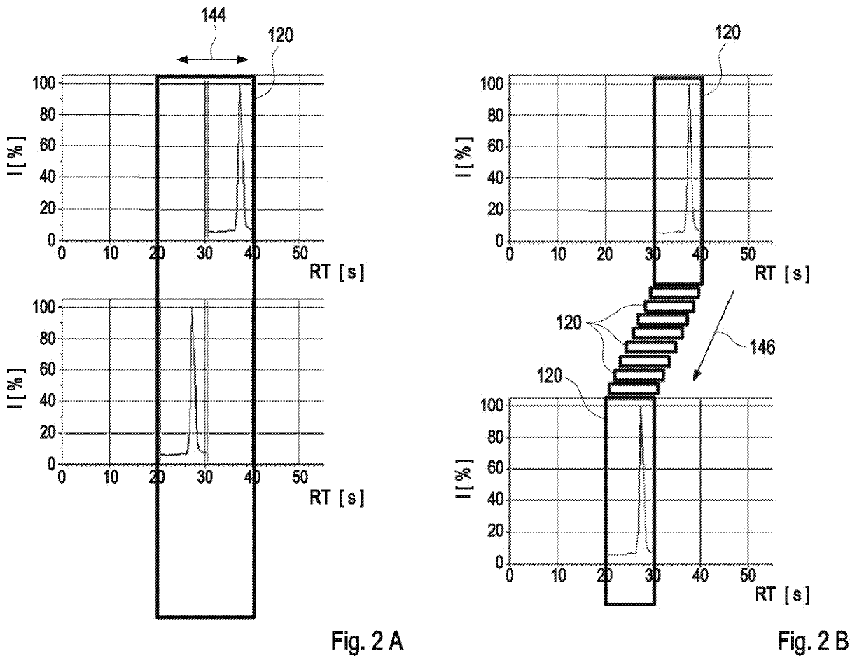

The method comprises in step b) (denoted with reference number 118) determining at least one reference peak information of the transition of the analyte using an initial setting of a measurement window 120, wherein the measurement window 120 is defined by a time frame of retention times. Examples of measurement windows 120 are shown in FIG. 2 B.

The reference peak information may be at least one information of a peak, i.e., a local maximum, of the chromatogram corresponding and/or relating to the analyte of interest of the reference measurement which is suitable to limit the relevant time frame for measurement of the analyte of interest. The reference peak information may comprise one or more of: peak maximum, retention time, peak start time, peak end time, peak width, in particular the full width half maximum, peak shape, tailing factor, and/or any type of peak fitting and filtering. The determining of the reference peak information may comprise evaluating 122 the reference measurement. The evaluating 122 may comprise performing at least one data analysis comprising performing at least one peak finding algorithm and/or performing at least one peak fitting algorithm. The evaluating may comprise one or more of checking of raw data, preprocessing, smoothening, background reduction or removal, peak detection, peak integration.

The measurement window 120 is defined by a time frame of retention times. Limiting the measurement of the analyte of interest to a certain pre-defined time frame is generally known. The setting of the measurement window 120 may comprise values for one or both limits of the measurement window 120. Specifically, the setting of the measurement window 120 may comprise one or both of a value for a lower limit of the measurement window, i.e., a retention time at which the measurement starts, and a value for an upper limit of the measurement window, i.e., a retention time at which the measurement stops.

The initial setting may be a setting of the measurement window 120 which is used for determining the reference peak information. The initial setting may be pre-determined and/or pre-defined. For example, in case of a first measurement after change of a column of the LC device, the initial setting may be a default setting which may be deposited in the data base. The method may comprise at least one initial calibration step, not shown here, wherein in the initial calibration step the reference measurement and/or the initial setting may be determined. The initial calibration step may be performed during and/or subsequent to at least one quality control run of the liquid chromatography mass spectrometry device and/or during and/or subsequent to at least one internal standard samples run. A start of the initial calibration step may be triggered by changing of a column of the liquid chromatography mass spectrometry device 111 and/or after a predefined or pre-determined time and/or after a predefined or predetermined number of runs, or other suitable counters. In case of performing the method repeatedly, the initial setting may be a setting of the measurement window 120 determined from the measurement of at least one prior run or a plurality of prior runs, such as a mean value for the limits of the time frame.

The method furthermore comprises in step c) determining an actual setting of the measurement window 120 considering the reference peak information, wherein the determining comprises adjusting the time frame. The actual setting may be a setting of the measurement window 120 determined by considering the reference peak information determined in step b). In addition, to considering the reference peak information of only the preceding measurement, the actual setting may be determined considering the preceding measurement or a plurality of preceding measurements. The determining of the actual setting may comprise evaluating the reference peak information and thereby determining a lower and/or an upper limit of the measurement window. Specifically, at least one automated analysis of retention times may be performed, denoted with reference number 124. Moreover, the measurement window may be automatically reassigned, denoted with reference number 126. The actual setting may be determined and/or calculated based on expected retention time and tailing factor. The actual setting may be determined by comparing peak, in particular signal intensity, and background. For example, signal intensity and background may be compared by defining and/or using at least one threshold value at which the peak starts and/or at least one threshold value at which the peak ends. The actual setting may be determined by making a prediction based on a plurality of datasets.

The initial setting may comprise a broader time frame of retention times compared to the actual setting. Specifically, the initial setting of the measurement window 120 may be selected so broad such that it is ensured that the peak corresponding to the analyte of interest lies within the time frame. Subsequently the initial setting of the measurement window 120 may be optimized in view of measurement results which allows for reducing width of the measurement window and/or for positioning the measurement window 120, specifically to take into account changes of the LC column such as due to aging or other changes. The reference measurement may comprise or may be a known MRM transition, wherein the method comprises optimizing their measurement. The determining of the actual setting may comprise adapting and/or changing the initial setting of the measurement window depending on at least one subsequent measurement. The timing of the measurement may be fixed. However, due to variation of the peak position as a result of ongoing analysis, specifically in case of repeatedly performing method steps a) to d), the retention time may shift and may be adapted based on prior measurements. The method may comprise adjustment of the MRM scheduled timing as a function of changing parameters over time. Thus, the optimized retention time may be used to optimize scheduled MRM measurements and thus freeing up time for more MRM transitions.

The method comprises in step d) measuring, denoted with reference number 130, the transition of the analyte within a sample with the liquid chromatography mass spectrometry device. The measurement may be triggered 128 by a user e.g., by entering at least one input to at least one human-machine-interface of the liquid chromatography mass spectrometry device 111.

The method further comprises in step d) determining a measured peak information, denoted with reference number 132, of the transition of the analyte using the actual setting of the measurement window 120. The measured peak information may be at least one information of a peak of the chromatogram measured in step d) corresponding and/or relating to the analyte of interest using the actual setting of the measurement window 120. The measured peak information comprises one or more of: peak maximum, retention time, peak start time, peak end time, peak width, in particular the full width half maximum, peak shape, tailing factor, and/or any type of peak fitting and filtering. The determining of the measured peak information may comprise evaluating the actual measurement. The evaluating may comprise performing at least one data analysis comprising performing at least one peak finding algorithm and/or performing at least one peak fitting algorithm.

The method further may comprise updating 134 the data set 112 by adding the measurement of the transition of the analyte to the data set 112. Specifically, in case the method steps a) to d) are performed repeatedly, the data set 112 may be updated after performing step d) such that the subsequent step a) is performed using an updated initial setting. Thus, the updating 134 may be performed permanently.

Method steps a) to d) may be performed repeatedly. In step a) the most recent measurement may be used as reference measurement. In step b) the reference peak information of the transition of the analyte may be determined using the most recently determined actual setting as initial setting. After a plurality of repetitions of methods steps a) to d), in step a) a mean value of a plurality of preceding measurements may be used as reference measurement. The mean value may be determined by using at least five preceding measurements. Additionally or alternatively, in particular in case of larger changes between runs, a moving average of a plurality of preceding measurements may be used as reference measurement. Additionally or alternatively, a maximum amount of preceding measurements for calculating the mean value may be limited. Using more than one measurement may ensure that the reference data is corrected with respect to outliers or sample influence.

A width of the measurement window 120 may narrow with number of repetitions. Thus, the measurement window 120 becomes even better and/or more beneficial for clearing up more MS detection window. The detection window may be a time frame in which the mass spectrometry device has to perform a measurement of a sample.

FIG. 1 B shows a further flowchart of the method according to the present disclosure, wherein, in addition to the embodiment shown in FIG. 1 A, the method may further comprise at least one in-situ adjustment step 136. The in-situ adjustment step 136 may be performed during step d). In the in-situ adjustment step 136 intensity of the transition of the analyte may be monitored during the measurement and compared to at least one predetermined or predefined threshold level. The liquid chromatography mass spectrometry device 111 may comprise at least one further data base 138 configured for storing at least one definition of threshold levels and/or threshold values. For example, the at least one threshold level and/or the at least one threshold value may be defined by percentual change of signal intensity to background. Additionally or alternatively, at least one absolute value may be used as threshold for determining exceedance or undershoot. The further data base 138 may be configured to receive (denoted with reference number 142) input information from the data base 114 such as values for the threshold levels. Thus, the threshold levels may be data driven. If the intensity falls below the predefined threshold level acquisition of the transition may be stopped. The predetermined or predefined threshold level may be defined by a factor X times the signal-to-noise ratio which is also known from the start. The in-situ adjustment step may be implemented as a feedback loop 140 with automated live adjustment of measurement parameter. For example, a measurement, i.e., a specific MRM, may start at a time known on the basis of previous measurements. A run time of the measurement may be determined on the basis of an actual measured intensity of this MRM. In case the intensity falls below the predetermined or predefined threshold level the acquisition of that MRM may be stopped and may allow to free dwell time for other MRMs running at the same time. This approach may be reliable for well articulated peaks. However, problems may arise when signal to noise is low, e.g., a small peak with high variation in individual signal. For these cases it may be advantageous that the predetermined or predefined threshold level may be given by an end of fit curve such as a Gaussian curve. The fit curve may allow defining the peak region such as start of the peak and end of the peak. In particular, a certain peak height at the end of the peak, denoted as end of the fit curve, may be used as threshold level. In principle, use of a fit curve during measurement is problematic since the full signal is not present at this stage. However, a fit curve such as a Gaussian curve can be used since the parameters of the Gaussian or other fit curve may be determined from the one or more preceding measurements. This may allow determining all parameters of the fit curve with only a single iteration. Additionally or alternatively, the fit curve may be determined from a measurement of an internal standard. This may allow accelerating the fitting procedure. Additionally or alternatively, a combination of fit results of different peaks of the same analyte may be used to enhance robustness of the fit result. The fit curve, in particular of a Gaussian, may be independent or less dependent from background and, thus, advantageous even at high noise.

The method steps b) to c) and in step d) the determining of the measured peak information of the transition of the analyte may be performed by at least one computer. Specifically, the method steps b) to c) and in step d) the determining of the measured peak information of the transition of the analyte may be performed fully automatically. The method specifically may fully or partially be computer-implemented, specifically on a computer of a device for multiple transition monitoring, such as a processor.

FIGS. 2 A and 2 B show a comparison of a usual approach, shown in FIG. 2 A, and the method according to the present disclosure, shown in FIG. 2 B, such as described with respect to FIGS. 1 A and 1 B. Specifically, intensity I in % as a function of retention time RT in seconds is depicted. In FIGS. 2 A and 2 B, the upper plot shows the chromatogram for a new LC column and/or initial conditions and the lower plot shows the chromatogram for an aged LC column and/or potential other changes in the experimental performance of the system. For the usual approach shown in FIG. 2 A large measurement windows are used in order to compensate potential changes (denoted with arrow 144) over lifetime of the LC column. For example, the width Δt of the measurement window may be Δt=20 s and the position of the measurement window may be from 20 to 40 s. In FIG. 2 B the measurement window 120 can be selected narrower compared to FIG. 2 A, e.g., Δt=10 s. Moreover, the measurement window 120 can be maintained constant or can be even narrowed due to repeating method steps a) to d) and permanent reassignment (denoted with arrow 146) of the measurement window 120 based on prior measurements and in-situ adjustment.

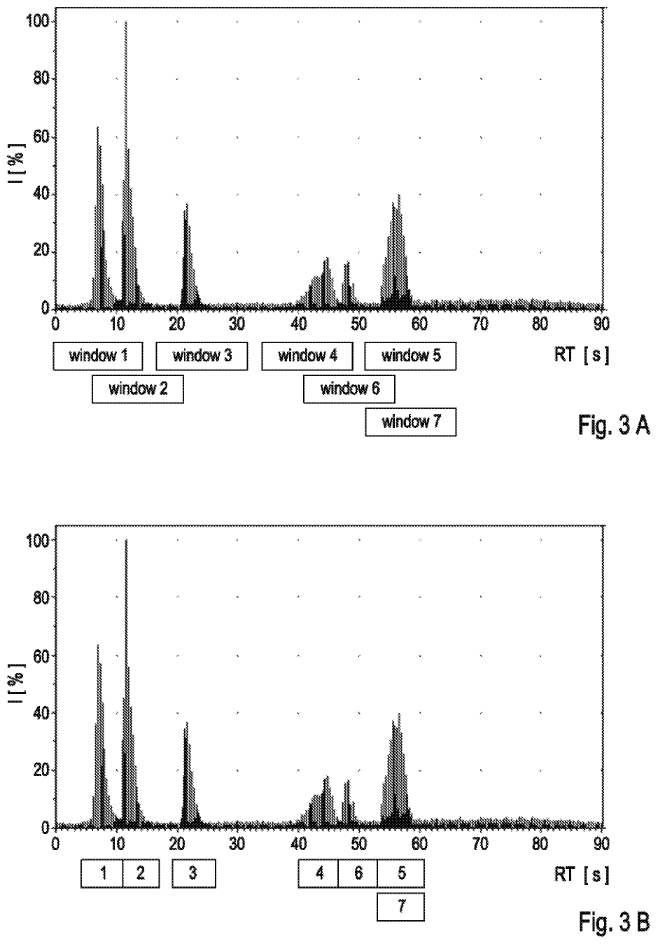

FIGS. 3 A and 3 B show a further comparison of measurement windows of a usual approach, shown in FIG. 3 A and of the method according to the present disclosure, shown in FIG. 3 B. Specifically, intensity I in % as a function of retention time RT in seconds is depicted. Moreover, in FIGS. 3 A and 3 B seven measurement windows and their width are shown each corresponding to a respective peak in the chromatogram. For the usual approach shown in FIG. 3 A large measurement windows are used in order to compensate potential changes over lifetime of the LC column. In contrast, in FIG. 3 B the measurement windows 120 are narrowed compared to FIG. 3 A. Using such narrow measurement windows is possible due to permanent reassignment of the measurement window 120 based on prior measurements and in-situ adjustment. No safety margins are necessary. The measurements windows show less overlap. Higher sampling rate per peak is possible resulting in more points per peak.

FIG. 4 shows highly schematically a device 110 for multiple transition monitoring according to the present disclosure. The device 110 comprises the at least one liquid chromatography mass spectrometer device 111 configured for multiple transition monitoring. The liquid chromatography mass spectrometry device 111 may be or may comprise at least one high-performance liquid chromatography (HPLC) device or at least one micro liquid chromatography (μLC) device. The liquid chromatography mass spectrometry device 111 may comprise a liquid chromatography (LC) device and a mass spectrometry (MS) device, wherein the LC device and the MS are coupled via at least one interface. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analytes of interest. The LC column may be exchangeable, for example after a predefined or pre-determined time and/or number of runs, and/or other suitable counters. The mass spectrometry device may be or may comprise at least one quadrupole mass spectrometry device. The interface coupling the LC device and the MS may comprise at least one ionization source configured for generating of molecular ions and for transferring of the molecular ions into the gas phase.

The device 110 further comprises the data base 114 configured for storing the data set 112 comprising the at least one reference measurement 116 of at least one transition of at least one analyte. With respect to description of the data set 112 and data base 114 reference is made to the description of FIGS. 1 A and 1 B above.

The device 110 comprises at least one evaluation device 148. The evaluation device 148 is configured for determining at least one reference peak information of the transition of the analyte using an initial setting of the measurement window 120. The measurement window 120 is defined by a time frame of retention times. The evaluation device 148 is configured for determining an actual setting of the measurement window 120 considering the reference peak information. The determining comprises adjusting the time frame. The evaluation device 148 is configured for determining a measured peak information of the transition of the analyte of a subsequent measurement of the transition of the analyte with the liquid chromatography mass spectrometry device 111 using the actual setting of the measurement window 120.

### LIST OF REFERENCE NUMBERS

110 device for multiple transition monitoring
111 CMS device
112 data set
114 data base
116 reference measurement
118 step b)
120 measurement window
122 evaluating the reference measurement
124 automated analysis
126 reassigning measurement window
128 trigger
130 measurement transition
132 determining measured peak information
134 updating
136 in-situ adjustment step
138 further data base
140 feedback loop
142 receiving input
144 arrow
146 arrow
148 evaluation device

What is claimed is:

1. A method for multiple transition monitoring using a liquid chromatography mass spectrometry device, the method comprises the following steps:
determining at least one data set from at least one data base, the data set comprising at least one reference measurement of at least one transition of at least one analyte with the liquid chromatography mass spectrometry device;
determining at least one reference peak information of the transition of the analyte using an initial setting of a measurement window, wherein the initial setting is a setting of the measurement window which is used for determining the reference peak information, wherein the measurement window is defined by a time frame of retention times;
determining an actual setting of the measurement window considering the reference peak information, wherein the determining comprises adjusting the time frame, wherein the actual setting is a setting of the measurement window determined by considering the reference peak information determined in step b);
measuring the transition of the analyte with the liquid chromatography mass spectrometry device and determining a measured peak information of the transition of the analyte using the actual setting of the measurement window.

2. The method according to claim 1, wherein the method further comprises updating the data set by adding the measurement of the transition of the analyte to the data set.

3. The method according to claim 2, wherein method steps a) to d) are performed repeatedly, wherein in step a) the most recent measurement is used as reference measurement, wherein in step b) the reference peak information of the transition of the analyte is determined using the most recently determined actual setting as initial setting.

4. The method according to claim 2, wherein in step a) one or both of a mean value or a moving average of a plurality of preceding measurements is used as reference measurement.

5. The method according to claim 1, wherein the method further comprises at least one in-situ adjustment step, wherein the in-situ adjustment step is performed during step d), wherein in the in-situ adjustment step intensity of the transition of the analyte is monitored during the measurement and compared to at least one predetermined or predefined threshold level, wherein if the intensity falls below the threshold level acquisition of the transition is stopped.

6. The method according to claim 1, wherein one or both of the reference peak information or the measured peak information comprise one or more of: peak maximum, retention time, peak start time, peak end time, peak width peak shape, tailing factor, or any type of peak fitting and filtering.

7. The method according to claim 6, wherein one or both of the reference peak information or the measured peak information comprise the full width half maximum.

8. The method according to claim 1, wherein the method comprises at least one initial calibration step, wherein in the initial calibration step one or both of the reference measurement or the initial setting are determined.

9. The method according to claim 8, wherein the initial calibration step is one or more of performed during or subsequent to at least one quality control run of the liquid chromatography mass spectrometry device, performed during or subsequent to at least one internal standard samples run.

10. The method according to claim 8, wherein a start of the initial calibration step is triggered by one or more of changing of a column of the liquid chromatography mass spectrometry device after a predefined or predetermined time or after a predefined or predetermined number of runs, or other suitable counters.

11. The method according to claim 1, wherein the initial setting comprises a broad time frame of retention times compared to the actual setting.

12. The method according to claim 1, wherein the method comprises repeating method steps a) to d), wherein a width of the measurement window narrows with number of repetitions.

13. The method according to claim 1, wherein the reference measurements and the measuring of the transition of the analyte are performed under substantially the same conditions.

14. The method according to claim 1, wherein the method steps b) to c) and in step d) the determining of the measured peak information of the transition of the analyte are performed by at least one computer.

15. A device for multiple transition monitoring of at least one analyte in a sample comprising:
at least one liquid chromatography mass spectrometer device configured for multiple transition monitoring;
at least one data base configured for storing at least one data set comprising at least one reference measurement of at least one transition of at least one analyte;
at least one evaluation device, wherein the evaluation device is configured for determining at least one reference peak information of the transition of the analyte using an initial setting of a measurement window, wherein the initial setting is a setting of the measurement window which is used for determining the reference peak information, wherein the measurement window is defined by a time frame of retention times, wherein the evaluation device is configured for determining an actual setting of the measurement window considering the reference peak information, wherein the determining comprises adjusting the time frame, wherein the actual setting is a setting of the measurement window determined by considering the determined reference peak information, wherein the evaluation device is configured for determining a measured peak information of the transition of the analyte of a subsequent measurement of the transition of the analyte with the liquid chromatography mass spectrometry device using the actual setting of the measurement window.
